(19) United States
(12) Patent Application Publication
(10) Pub. No.: US 2026/0210974 A1
(43) Pub. Date: Jul. 23, 2026

(54) METHODS OF PROGNOSIS FOR HEMORRHAGIC TRANSFORMATION FOLLOWING ENDOVASCULAR TREATMENT

(71) Applicants: The Board of Trustees of the Leland Stanford Junior University, Stanford, CA (US); ROTHSCHILD FOUNDATION HOSPITAL, Paris (FR); UNIVERSITÉ PARIS-CITÉ, Paris (FR); CENTRE HOSPITALIER UNIVERSITAIRE DE TOULOUSE, Toulouse (FR); UNIVERSITE TOULOUSE III - PAUL SABATIER, Toulouse (FR); INSTITUT NATIONAL DE LA SANTÉ ET DE LA RECHERCHE MÉDICALE, Paris (FR); FONDATION HÔPITAL SAINT-JOSEPH, PARIS (FR)

(72) Inventors: Brice L. Gaudilliere, Palo Alto, CA (US); Amy Tsai, Redwood City, CA (US); Julien Hedou, San Francisco, CA (US); Mikael Mazighi, Redwood City, CA (US); Jean-Marc Olivot, Redwood City, CA (US); Jean-Philippe Desilles, Paris (FR); Benoit Ho-Tin-Noe, Ivry Sur Seine (FR); Benjamin Maier, Charenton-Le-Pont (FR)

(21) Appl. No.: 19/140,993

(22) PCT Filed: Dec. 21, 2023
(86) PCT No.: PCT/US2023/085562

§ 371 Date: Jun. 18, 2025

Related U.S. Application Data
(60) Provisional application No. 63/434,712, filed on Dec. 22, 2022.

(51) Int. Cl. G01N 33/68; G01N 15/14; G01N 33/50; G01N 33/53

(52) U.S. Cl. CPC G01N 33/6893; G01N 15/14; G01N 33/5091; G01N 33/5094; G01N 33/53; G01N 2333/4703; G01N 2458/15; G01N 2800/2871; G01N 2800/50; G01N 2800/60; G01N 2800/7019

(57) ABSTRACT

Methods are provided for classification, diagnosis, prognosis, theranosis, and/or prediction of an outcome following endovascular treatment (EVT) in an individual suffering from an acute ischemic stroke with respect to development of hemorrhagic transformation (HT). The data and integrated model provided herein demonstrates a pre-operative assessment of single-cell biomarkers for accurate prediction of HT following EVT in individuals suffering from acute ischemic strokes. The integrated model incorporates “immune features” such as activation of signaling proteins and/or the frequency of specific cell subset(s). The analysis and prediction of HT is used to guide therapeutic approaches to EVT and the post-treatment care. Specifically, the level of certain features (e.g. elevated prpS6 in memory Th1 CD4+T cells, elevated pCREB in naïve Th1 CD4+T cells and increased frequency of non-classical monocytes) demonstrate an increased likelihood of HT occurring following EVT.

### CROSS REFERENCE TO RELATED APPLICATIONS

Pursuant to 35 U.S.C. § 119 (e), this application claims priority to the filing date of U.S. Provisional Patent Application Ser. No. 63/434,712 filed Dec. 22, 2022, the disclosure of which application is herein incorporated by reference.

### GOVERNMENT RIGHTS

This invention was made with Government support under contract GM137936 awarded by the National Institutes of Health. The Government has certain rights in the invention.

### BACKGROUND OF THE INVENTION

Acute ischemic stroke (AIS) is one of the leading cause of morbidity and mortality worldwide, resulting in significant economic cost due to more frequent hospitalizations in care facilities, earlier retirement, and greater use of socioeconomic resources. Despite the 90% recanalisation rate after endovascular treatment (EVT), over 50% of patients with AIS suffer hemorrhagic transformation (HT), the major modifiable predictor of poor functional recovery. However, there is a lack of biological and clinical biomarkers for accurate prediction of HT after AIS, an essential prerequisite for the development individualized patient-centered care. Emerging preclinical evidence suggests that dysfunctional inflammatory processes resulting in persistent microcirculation thrombosis and blood brain barrier leakage are critical to the pathogenesis of HT. As such, high-dimensional immune profiling of peripheral blood inflammatory events is a powerful approach to identify mechanistic and easily accessible predictive biomarkers for risk of HT after an EV treatment for AIS.

The present disclosure provides methods for the prognosis of hemorrhagic transformation following endovascular treatment.

### SUMMARY OF THE INVENTION

Methods are provided for classification, diagnosis, prognosis, theranosis, and/or prediction of an outcome following endovascular treatment (EVT) in an individual suffering from an acute ischemic stroke with respect to the development of hemorrhagic transformation (HT). The data and integrated model provided herein demonstrate a pre-operative assessment of single-cell biomarkers for accurate prediction of HT following EVT in individuals suffering from acute ischemic strokes. The integrated model incorporates “immune features” such as activation of signaling proteins (e.g., pSTAT1, 3, 5, 6, pERK, p38, pMK2, prpS6, pCREB, pNF-_{κ}B, total I_{κ}B, etc.) and/or the frequency of specific cell subset(s) (e.g. classical and non-classical monocytes, natural killer cells, neutrophils, dendritic cells, T cells, T helper cells, etc.). The analysis and prediction of HT are used to guide therapeutic approaches to EVT and post-treatment care. Specifically, the level of certain features (e.g. elevated prpS6 in memory Th1 CD4+ T cells, elevated pCREB in naïve Th1 CD4+ T cells, and increased frequency of non-classical monocytes) demonstrate an increased likelihood of HT occurring following EVT.

Provided herein are methods for assessing the propensity to develop hemorrhagic transformation (HT) for an individual suffering from an acute ischemic stroke following an endovascular treatment (EVT). In some embodiments, the methods comprise obtaining a cellular biological sample for analysis comprising immune cells from a patient after having the acute ischemic stroke, measuring single-cell levels of activated signaling proteins in immune cell subset(s); determining whether levels of activated signaling proteins associated with propensity to develop hemorrhagic transformation are present; and providing an assessment of the patient's prognosis for propensity to develop hemorrhagic transformation after the endovascular treatment.

The endovascular treatment may be any treatment deemed useful in treating the acute ischemic stroke. In some embodiments, the endovascular treatment is a thrombectomy. In some embodiments, the endovascular treatment is a thrombolysis.

A cellular biological sample for use in the methods disclosed herein may be any cellular biological sample comprising the immune cell subset(s) of interest. In some embodiments, the cellular biological is a blood sample. In some embodiments, the blood sample is a venous blood sample or an arterial blood sample. In some embodiments, the blood sample is a veinous blood sample or arterial blood sample near the site of the acute ischemic stroke (i.e. near a thrombus).

The obtaining of the cellular biological sample may be performed at any time following the acute ischemic stroke. In some embodiments, the obtaining is performed prior to the endovascular treatment. In some embodiments, the obtaining is performed after the endovascular treatment. In some embodiments, the obtaining is performed before and after the endovascular treatment. When the obtaining is performed before and after the endovascular treatment, the cellular biological samples are measured and the results of the measuring may be compared to one another, where the results of the measuring performed on the cellular biological sample collected before the endovascular treatment is used as a reference for the measuring performed on the cellular biological samples collected after the therapy. The comparison may be used to determine if there is an increase or decrease in the level of signaling protein activation or in the frequency of one or more specific immune cell subset(s).

The methods of the present disclosure involve measuring single cell levels of activated signaling proteins in immune cell subset(s). The measuring may comprise any non-invasive method of quantifying activated signaling proteins in the immune cell subset(s) in the cellular biological sample. In general, the measuring comprises physically contacting cells with a panel of affinity reagents specific for activated signaling proteins and for markers that distinguish subsets of immune cells. Usually, the affinity reagents comprise a detectable label, e.g. isotope, fluorophore, etc. The signal intensity of the markers is measured, preferably at a single-cell level. Suitable methods of measuring include, without limitation, flow cytometry, mass cytometry, confocal microscopy, and the like. The data, which can include measurements of the intensity of signaling molecules and phosphorylation status in selected immune cell subsets, etc., is compared to measurements of the same from the reference population. The reference may be obtained from a normal control, a pre-determined level obtained from one or a population of individuals, from a negative control for ex vivo activation, and the like. The data can be normalized for comparison.

The measuring may be gated on Th1 CD4+ T cells or subsets of Th1 CD4+ T cells, including, without limitation, memory Th1 CD4+ T cells, naive Th1 CD4+ T cells, etc. In some embodiments, the measuring is also gated on neutrophils. The measuring may include, without limitation, mass spectroscopy, protein assays (aptamer-based or antibody-based detection of proteins), high-dimensional mass cytometry, fluorescence-based flow cytometry immunoassay, etc.

In the methods disclosed herein, activated signaling protein levels in immune cell subset(s) are assessed at one or more timepoints to predict the likelihood of HT following EVT in individuals suffering from an acute ischemic stroke. Signal protein activation may be the activation of specific proteins involved in key signaling processes in immune cells, such as, for example, mTOR signaling. A protein may be activated when there is a change in the phosphorylation status, conformation state or cleavage at specific residues. For example, activation of CREB protein may be manifest by the presence of phosphorylated CREB (i.e. pCREB). The changes in signal protein activation are identified using specific regents that are able to differentiate between activated and non-activated states (e.g. an antibody). The specific activated signaling proteins that are used in the methods disclosed herein include, without limitation, cleaved PARP (cPARP), pCREB, pSTAT5, pp38, pSTAT1, pSTAT3, prpS6, pMAPKAPK2, Tbet, Ki67, FoxP3, IκB, pNFκB, pERK1/2, pSTAT6, etc. In some embodiments, the signaling proteins of interest comprise pCREB and prpS6.

The signaling proteins may be activated in all immune cell subsets or one or more specific immune cell subsets. Immune cell subsets of interest include both innate and adaptive immune cell subsets. Immune cell subsets that find use in the present disclosure include without limitation, CCR2− nonclassical monocytes, CCR2+ classical monocytes, CD56brightCD16-natural killer (NK) cells, CD56dimCD16+ NK cells, CD62L− aged neutrophils, CD62L+ immature neutrophils, intermediate monocytes, myeloid dendritic cells, plasmacytoid dendritic cells, myeloid-derived suppressor cells, CD4+ central memory T cells, CD4+ effector memory T cells, CD4+ naive T cells, CD4+ resident memory T cells, CD8+ central memory T cells, CD8+ effector memory T cells, CD8+ naive T cells, CD8+ resident memory T cells, gamma-delta T cells, natural-killer T cells, memory T helper 1 (Th1) cells, naive Th1 cells, memory regulatory T cells, and naive regulatory T cells, etc.

In some embodiment, increased prpS6 signal protein activation in memory Th1 CD4+ T cells and neutrophils is indicative of increased likelihood for an individual to have a HT following EVT. In some embodiment, increased pCREB signal protein activation in naive Th1 CD4+ T cells is indicative of increased likelihood for an individual to have a HT following EVT.

In addition to measuring the single cell levels of activated signaling proteins, the methods may further comprise measuring the frequency of specific immune cell subtypes may also be indicative of an individual's likelihood to develop HT following EVT. The frequency of a specific immune cell subtype may be high or low relative to a reference. The reference may be obtained from a normal control, a pre-determined level obtained from one or a population of individuals, from a negative control for ex vivo activation, and the like. In a preferred embodiment, a high or increased frequency of non-classical monocytes is indicative of increased likelihood for an individual to have a HT following EVT. In some embodiments, the combination of increased prpS6 signal protein activation in memory Th1 CD4+ T cells and neutrophils, creased pCREB signal protein activation in naive Th1 CD4+ T cells, and a high or increased frequency of non-classical monocytes is indicative of increased likelihood for an individual to have a HT following EVT.

An aspect of the method is determining whether changes in the levels of activated signaling proteins associated with the propensity to develop HT are present. The determining may be performed by integrating the results of the measuring step into a multivariate model. In some embodiments, the level of prpS6 in memory Th1 CD4+ T cells is integrated into the multivariate model and a score is outputted that is indicative of whether or not the individual is at increased risk of having HT following EVT. In some embodiments, the level of pCREB in naive Th1 CD4+ T cells is integrated into the multivariate model and a score is outputted that is indicative of whether or not the individual is at increased risk of having HT following EVT. In some embodiments, the frequency of non-classical monocytes is integrated into the multivariate model and a score is outputted that is indicative of whether or not the individual is at increased risk of having HT following EVT. In some embodiments, the level of prpS6 in memory Th1 CD4+ T cells, the level of pCREB in naive Th1 CD4+ T cells, and the frequency of non-classical monocytes are integrated into the multivariate model and a score is outputted that is indicative of whether or not the individual is at increased risk of having HT following EVT.

Individuals showing immune features indicative of HT can be treated accordingly. Because the diagnosis can be provided significantly before clinical symptoms or the occurrence of HT, the methods herein provide a means of timely intervention. In addition, the results provided by the methods with assist in the determination of the timing of the endovascular treatment, the use of immune-modifying therapies in addition to the EVT in order to prevent the occurrence of HT following a determination of increased likelihood of HT following EVT, and will assist in the determination of timing and disposition of discharge from the hospital and clinical follow up.

Also described herein is a method for assessing prognosis for HT following EVT, comprising: obtaining a dataset associated with a sample obtained from the individual suffering from the acute ischemic stroke, wherein the dataset comprises quantitative data for specific immune cell subset and the activated signaling proteins contained therein; and analyzing the dataset for changes at the single cell level for these markers, wherein a statistically significant match with a HT pattern is indicative of the prognosis to develop HT following EVT. The data may be analyzed by a computer processor. The processor may be communicatively coupled to a storage memory for analyzing the data. Also described herein is a computer-readable storage medium storing computer-executable program code, the program code comprising: a program code for storing and analyzing data obtained by the methods of the invention.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1A discloses the design of the study used to generate the multivariate model.

FIG. 1B discloses the performance of the multivariate model for the determining the propensity of an individual to develop hemorrhagic transformation following endovascular treatment. The multivariate model was built on 324 input variables derived from the single-cell assessment of 47 proteomic parameters, and including the frequency of 27 cell subsets and the median intracellular activity of 11 signaling proteins.

### DETAILED DESCRIPTION

These and other features of the present teachings will become more apparent from the description herein. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Most of the words used in this specification have the meaning that would be attributed to those words by one skilled in the art. Words specifically defined in the specification have the meaning provided in the context of the present teachings as a whole, and as are typically understood by those skilled in the art. In the event that a conflict arises between an art-understood definition of a word or phrase and a definition of the word or phrase as specifically taught in this specification, the specification shall control.

It must be noted that, as used in the specification and the appended claims, the singular forms “a,” “an,” and “the” include plural referents unless the context clearly dictates otherwise.

Compositions and methods are provided for prognostic classification of patients following an acute ischemic stroke and endovascular treatment for their propensity to develop hemorrhagic transformation, using an analysis at the single cell level of activation of signaling pathways in specific immune cell subsets. Patterns of response are obtained by quantitating specific activated signal proteins in immune cell subsets of interest, before and/or after endovascular treatment. The pattern of response is indicative of the patient's propensity to develop hemorrhagic transformation. Once a classification or prognosis has been made, it can be provided to a patient or caregiver. The classification can provide prognostic information to guide clinical decision making, both in terms of institution of and escalation of treatment, and in some cases may further include selection of a therapeutic agent or regimen.

The information obtained from the activated signaling protein patterns of response can be used to (a) determine type and level of therapeutic intervention warranted and (b) to optimize the selection of therapeutic agents. With this approach, therapeutic regimens can be individualized and tailored according to the propensity to develop hemorrhagic transformation, thereby providing a regimen that is individually appropriate.

The terms “subject,” “individual,” and “patient” are used interchangeably herein to refer to a vertebrate, preferably a mammal, more preferably a human. Mammalian species that provide samples for analysis include canines; felines; equines; bovines; ovines; etc. and primates, particularly humans. Animal models, particularly small mammals, e.g. murine, lagomorpha, etc. can be used for experimental investigations. The methods of the invention can be applied for veterinary purposes.

As used herein, the term “theranosis” refers to the use of results obtained from a diagnostic method to direct the selection of, maintenance of, or changes to a therapeutic regimen, including but not limited to the choice of one or more therapeutic agents, changes in dose level, changes in dose schedule, changes in mode of administration, and changes in formulation. Diagnostic methods used to inform a theranosis can include any that provides information on the state of a disease, condition, or symptom.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” refers to the amount of an agent that is sufficient to effect beneficial or desired results. The therapeutically effective amount will vary depending upon the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose will vary depending on the particular agent chosen, the dosing regimen to be followed, whether it is administered in combination with other compounds, timing of administration, the tissue to be imaged, and the physical delivery system in which it is carried.

“Suitable conditions” shall have a meaning dependent on the context in which this term is used. That is, when used in connection with an antibody, the term shall mean conditions that permit an antibody to bind to its corresponding antigen. When used in connection with contacting an agent to a cell, this term shall mean conditions that permit an agent capable of doing so to enter a cell and perform its intended function. In one embodiment, the term “suitable conditions” as used herein means physiological conditions.

The term “inflammatory” response is the development of a humoral (antibody mediated) and/or a cellular response, which cellular response may be mediated by antigen-specific T cells or their secretion products), and innate immune cells. An “immunogen” is capable of inducing an immunological response against itself on administration to a mammal or due to autoimmune disease.

The terms “biomarker,” “biomarkers,” “marker” or “markers” for the purposes of the invention refer to, without limitation, proteins together with their related metabolites, mutations, variants, polymorphisms, modifications, fragments, subunits, degradation products, elements, and other analytes or sample-derived measures. Markers can include expression levels of an intracellular protein or extracellular protein. Markers can also include combinations of any one or more of the foregoing measurements, including temporal trends and differences. Broadly used, a marker can also refer to an immune cell subset.

To “analyze” includes determining a set of values associated with a sample by measurement of a marker (such as, e.g., presence or absence of a marker or constituent expression levels) in the sample and comparing the measurement against measurement in a sample or set of samples from the same subject or other control subject(s). The markers of the present teachings can be analyzed by any of various conventional methods known in the art. To “analyze” can include performing a statistical analysis, e.g. normalization of data, determination of statistical significance, determination of statistical correlations, clustering algorithms, and the like.

A “sample” in the context of the present teachings refers to any biological sample that is isolated from a subject, generally a sample comprising circulating immune cells. A sample can include, without limitation, an aliquot of body fluid, whole blood, PBMC (white blood cells or leucocytes), tissue biopsies, synovial fluid, lymphatic fluid, ascites fluid, and interstitial or extracellular fluid. “Blood sample” can refer to whole blood or a fraction thereof, including blood cells, white blood cells or leucocytes. Samples can be obtained from a subject by means including but not limited to venipuncture, biopsy, needle aspirate, lavage, scraping, surgical incision, or intervention or other means known in the art.

Optionally samples are activated ex vivo, which as used herein refers to the contacting of a sample, e.g. a blood sample or cells derived therefrom, outside of the body with a stimulating agent. In some embodiments whole blood is preferred. The sample may be diluted or suspended in a suitable medium that maintains the viability of the cells, e.g. minimal media, PBS, etc. The sample can be fresh or frozen. Stimulating agents of interest include those agents that activate T cells, e.g. LPS (1 μg/mL) and/or IFN-α (100 ng/ml). Generally the activation of cells ex vivo is compared to a negative control, e.g. medium only, or an agent that does not elicit activation. The cells are incubated for a period of time sufficient for activation. For example, the time for action can be up to about 1 hour, up to about 45 minutes, up to about 30 minutes, up to about 15 minutes, and may be up to about 10 minutes or up to about 5 minutes. In some embodiments the period of time is up to about 24 hours. Following activation, the cells are fixed for analysis.

A “dataset” is a set of numerical values resulting from evaluation of a sample (or population of samples) under a desired condition. The values of the dataset can be obtained, for example, by experimentally obtaining measures from a sample and constructing a dataset from these measurements; or alternatively, by obtaining a dataset from a service provider such as a laboratory, or from a database or a server on which the dataset has been stored. Similarly, the term “obtaining a dataset associated with a sample” encompasses obtaining a set of data determined from at least one sample. Obtaining a dataset encompasses obtaining a sample, and processing the sample to experimentally determine the data, e.g., via measuring antibody binding, or other methods of quantitating a signaling response. The phrase also encompasses receiving a set of data, e.g., from a third party that has processed the sample to experimentally determine the dataset.

“Measuring” or “measurement” in the context of the present teachings refers to determining the presence, absence, quantity, amount, or effective amount of a substance in a clinical or subject-derived sample, including the presence, absence, or concentration levels of such substances, and/or evaluating the values or categorization of a subject's clinical parameters based on a control, e.g. baseline levels of the marker.

Classification can be made according to predictive modeling methods that set a threshold for determining the probability that a sample belongs to a given class. The probability preferably is at least 50%, or at least 60% or at least 70% or at least 80% or a67t least 90% or higher. Classifications also can be made by determining whether a comparison between an obtained dataset and a reference dataset yields a statistically significant difference. If so, then the sample from which the dataset was obtained is classified as not belonging to the reference dataset class. Conversely, if such a comparison is not statistically significantly different from the reference dataset, then the sample from which the dataset was obtained is classified as belonging to the reference dataset class.

The predictive ability of a model can be evaluated according to its ability to provide a quality metric, e.g. AUC or accuracy, of a particular value, or range of values. In some embodiments, a desired quality threshold is a predictive model that will classify a sample with an accuracy of at least about 0.7, at least about 0.75, at least about 0.8, at least about 0.85, at least about 0.9, at least about 0.95, or higher. As an alternative measure, a desired quality threshold can refer to a predictive model that will classify a sample with an AUC (area under the curve) of at least about 0.7, at least about 0.75, at least about 0.8, at least about 0.85, at least about 0.9, or higher.

As is known in the art, the relative sensitivity and specificity of a predictive model can be “tuned” to favor either the selectivity metric or the sensitivity metric, where the two metrics have an inverse relationship. The limits in a model as described above can be adjusted to provide a selected sensitivity or specificity level, depending on the particular requirements of the test being performed. One or both of sensitivity and specificity can be at least about at least about 0.7, at least about 0.75, at least about 0.8, at least about 0.85, at least about 0.9, or higher.

“Affinity reagent”, or “specific binding member” may be used to refer to an affinity reagent, such as an antibody, ligand, etc. that selectively binds to a protein or marker of the invention. The term “affinity reagent” includes any molecule, e.g., peptide, nucleic acid, small organic molecule. For some purposes, an affinity reagent selectively binds to a cell surface marker, e.g. CD41, CD235ab, CD45, CD66, KIR, CD7, CD19, CD45RA, CD11b, CD4, CD8a, CD11c, CD123, TCRγδ, FceRIα, CD161, CD33, CRTH2, CD16, CD25, CD3, CXCR4, CD62L, CCR2, HLA-DR, CD14, CD56, CD61, and the like. For other purposes an affinity reagent selectively binds to a cellular signaling protein, particularly one which is capable of detecting an activation state of a signaling protein over another activation state of the signaling protein. Signaling proteins of interest include, without limitation, cPARP, pCREB, pSTAT5, pp38, pSTAT1, pSTAT3, prpS6, pMAPKAPK2, Tbet, Ki67, FoxP3, IκB, pNFκB, PERK1/2, pSTAT6, etc.

In some embodiments, the affinity reagent is a peptide, polypeptide, oligopeptide or a protein, particularly antibodies and specific binding fragments and variants thereof. The peptide, polypeptide, oligopeptide or protein can be made up of naturally occurring amino acids and peptide bonds, or synthetic peptidomimetic structures. Thus “amino acid”, or “peptide residue”, as used herein include both naturally occurring and synthetic amino acids. Proteins including non-naturally occurring amino acids can be synthesized or in some cases, made recombinantly; see van Hest et al., FEBS Lett 428: (1-2) 68-70 May 22, 1998 and Tang et al., Abstr. Pap Am. Chem. S218: U138 Part 2 Aug. 22, 1999, both of which are expressly incorporated by reference herein.

Methods of the present invention can be used to detect any particular signaling protein in a sample that is antigenically detectable and antigenically distinguishable from other signaling proteins which are present in the sample. For example, activation state-specific antibodies can be used to identify distinct signaling cascades of a subset or subpopulation of complex cell populations; and the ordering of protein activation (e.g., kinase activation) in potential signaling hierarchies. Hence, in some embodiments the expression and phosphorylation of one or more polypeptides are detected and quantified using methods of the present invention. As used herein, the term “activation state-specific antibody” or “activation state antibody” or grammatical equivalents thereof, refer to an antibody that specifically binds to a corresponding and specific antigen. Preferably, the corresponding and specific antigen is a specific form of a signaling protein. Also preferably, the binding of the activation state-specific antibody is indicative of a specific activation state of a specific signaling protein, e.g., a phosphorylated signaling protein.

The term “antibody” includes full length antibodies and antibody fragments, and can refer to a natural antibody from any organism, an engineered antibody, or an antibody generated recombinantly for experimental, therapeutic, or other purposes as further defined below. Examples of antibody fragments, as are known in the art, such as Fab, Fab′, F(ab′)2, Fv, scFv, or other antigen-binding subsequences of antibodies, either produced by the modification of whole antibodies or those synthesized de novo using recombinant DNA technologies. The term “antibody” comprises monoclonal and polyclonal antibodies. Antibodies can be antagonists, agonists, neutralizing, inhibitory, or stimulatory. They can be humanized, glycosylated, bound to solid supports, and possess other variations.

The antigenicity of an activated isoform of a signaling protein is distinguishable from the antigenicity of non-activated isoform of a signaling protein or from the antigenicity of an isoform of a different activation state. In some embodiments, an activated isoform of an element possesses an epitope that is absent in a non-activated isoform of an element, or vice versa. In some embodiments, this difference is due to covalent addition of moieties to an element, such as phosphate moieties, or due to a structural change in an element, as through protein cleavage, or due to an otherwise induced conformational change in an element which causes the element to present the same sequence in an antigenically distinguishable way. In some embodiments, such a conformational change causes an activated isoform of a signaling protein to present at least one epitope that is not present in a non-activated isoform, or to not present at least one epitope that is presented by a non-activated isoform of the element.

Many antibodies, many of which are commercially available (for example, see Cell Signaling Technology, www.cellsignal.com or Becton Dickinson, www.bd.com) have been produced which specifically bind to the phosphorylated isoform of a protein but do not specifically bind to a non-phosphorylated isoform of a protein. Many such antibodies have been produced for the study of signal transducing proteins which are reversibly phosphorylated. Particularly, many such antibodies have been produced which specifically bind to phosphorylated, activated isoforms of protein. Examples of proteins that can be analyzed with the methods described herein include, but are not limited to, NF-κB, CREB, ERK, p38, MK2, prpS6 and STAT1,3,5,6 or activated forms thereof.

The methods the invention may utilize affinity reagents comprising a label, labeling element, or tag. By label or labeling element is meant a molecule that can be directly (i.e., a primary label) or indirectly (i.e., a secondary label) detected; for example a label can be visualized and/or measured or otherwise identified so that its presence or absence can be known.

A compound can be directly or indirectly conjugated to a label which provides a detectable signal, e.g. non-radioactive isotopes, radioisotopes, fluorophores, enzymes, antibodies, particles such as magnetic particles, chemiluminescent molecules, molecules that can be detected by mass spec, or specific binding molecules, etc. Specific binding molecules include pairs, such as biotin and streptavidin, digoxin and anti-digoxin etc. Examples of labels include, but are not limited to, metal isotopes, optical fluorescent and chromogenic dyes including labels, fluorophores, label enzymes and radioisotopes. In some embodiments of the invention, these labels can be conjugated to the affinity reagents. In some embodiments, one or more affinity reagents are uniquely labeled.

Labels include optical labels such as fluorescent dyes or moieties. Fluorophores can be either “small molecule” fluors, or proteinaceous fluors (e.g. green fluorescent proteins and all variants thereof). In some embodiments, activation state-specific antibodies are labeled with quantum dots as disclosed by Chattopadhyay et al. (2006) Nat. Med. 12, 972-977. Quantum dot labeled antibodies can be used alone or they can be employed in conjunction with organic fluorochrome-conjugated antibodies to increase the total number of labels available. As the number of labeled antibodies increases so does the ability for subtyping known cell populations.

Activation state-specific antibodies can be labeled using chelated or caged lanthanides as disclosed by Erkki et al. (1988) J. Histochemistry Cytochemistry, 36:1449-1451, and U.S. Pat. No. 7,018,850. Other labels are tags suitable for Inductively Coupled Plasma Mass Spectrometer (ICP-MS) as disclosed in Tanner et al. (2007) Spectrochimica Acta Part B: Atomic Spectroscopy 62 (3): 188-195. Isotope labels suitable for mass cytometry may be used, for example as described in published application US 2012-0178183.

When using fluorescent labeled components in the methods and compositions of the present invention, it is recognized that different types of fluorescent monitoring systems, e.g., cytometric measurement device systems, can be used to practice the invention. In some embodiments, flow cytometric systems are used or systems dedicated to high throughput screening, e.g. 96 well or greater microtiter plates. Methods of performing assays on fluorescent materials are well known in the art and are described in, e.g., Lakowicz, J. R., Principles of Fluorescence Spectroscopy, New York: Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y.-L., San Diego: Academic Press (1989), pp. 219-243; Turro, N. J., Modern Molecular Photochemistry, Menlo Park: Benjamin/Cummings Publishing Col, Inc. (1978), pp. 296-361.

The detecting, sorting, measuring, or isolating step of the methods of the present invention can entail fluorescence-activated cell sorting (FACS) techniques, where FACS is used to select cells from the population containing a particular surface marker, or the selection step can entail the use of magnetically responsive particles as retrievable supports for target cell capture and/or background removal. A variety of FACS systems are known in the art and can be used in the methods of the invention (see e.g., WO99/54494, filed Apr. 16, 1999; U.S. Ser. No. 20010006787, filed Jul. 5, 2001, each expressly incorporated herein by reference).

In some embodiments, a FACS cell sorter (e.g. a FACSVantage™ Cell Sorter, Becton Dickinson Immunocytometry Systems, San Jose, Calif.) is used to sort and collect cells based on their activation profile (positive cells) in the presence or absence of an increase in activation level in an signaling protein in response to a modulator. Other flow cytometers that are commercially available include the LSR II and the Canto II both available from Becton Dickinson. See Shapiro, Howard M., Practical Flow Cytometry, 4th Ed., John Wiley & Sons, Inc., 2003 for additional information on flow cytometers.

In some embodiments, the cells are first contacted with labeled activation state-specific affinity reagents (e.g. antibodies) directed against specific activation state of specific signaling proteins. In such an embodiment, the amount of bound affinity reagent on each cell can be measured by passing droplets containing the cells through the cell sorter. By imparting an electromagnetic charge to droplets containing the positive cells, the cells can be separated from other cells. The positively selected cells can then be harvested in sterile collection vessels. These cell-sorting procedures are described in detail, for example, in the FACSVantage™ Training Manual, with particular reference to sections 3-11 to 3-28 and 10-1 to 10-17, which is hereby incorporated by reference in its entirety. See the patents, applications and articles referred to, and incorporated above for detection systems.

In some embodiments, the activation level of a signaling protein is measured using Inductively Coupled Plasma Mass Spectrometer (ICP-MS). An affinity reagent that has been labeled with a specific element binds to a marker of interest. When the cell is introduced into the ICP, it is atomized and ionized. The elemental composition of the cell, including the labeled affinity reagent that is bound to the signaling protein, is measured. The presence and intensity of the signals corresponding to the labels on the affinity reagent indicates the level of the signaling protein on that cell (Tanner et al. Spectrochimica Acta Part B: Atomic Spectroscopy, 2007 March; 62 (3): 188-195.).

Mass cytometry, e.g. as described in the Examples provided herein, finds use on analysis. Mass cytometry, or CyTOF (DVS Sciences), is a variation of flow cytometry in which antibodies are labeled with heavy metal ion tags rather than fluorochromes. Readout is by time-of-flight mass spectrometry. This allows for the combination of many more antibody specificities in a single samples, without significant spillover between channels. For example, see Bodenmiller at a. (2012) Nature Biotechnology 30:858-867.

STAT signaling pathways. In mammals seven members of the STAT family (STAT1, STAT2, STAT3, STAT4, STAT5a, STAT5b and STAT6) have been identified. JAKs contain two symmetrical kinase-like domains; the C-terminal JAK homology 1 (JH1) domain possesses tyrosine kinase function while the immediately adjacent JH2 domain is enzymatically inert but is believed to regulate the activity of JH1. There are four JAK family members: JAK1, JAK2, JAK3 and tyrosine kinase 2 (Tyk2). Expression is ubiquitous for JAK1, JAK2 and TYK2 but restricted to hematopoietic cells for JAK3.

STATs can be activated in a JAK-independent manner by src family kinase members and by oncogenic FLt3 ligand-ITD (Hayakawa and Naoe, Ann N Y Acad Sci. 2006 November; 1086:213-22; Choudhary et al. Activation mechanisms of STAT5 by oncogenic FLt3 ligand-ITD. Blood (2007) vol. 110 (1) pp. 370-4).

Strokes are a heterogeneous group of disorders involving sudden, focal interruption of cerebral blood flow that causes neurologic deficit. Strokes can be: Ischemic (80%), typically resulting from thrombosis or embolism, or Hemorrhagic (20%), resulting from vascular rupture (eg, subarachnoid hemorrhage, intracerebral hemorrhage). Transient stroke symptoms (typically lasting <1 hour) without evidence of acute cerebral infarction (based on diffusion-weighted MRI) are termed a transient ischemic attack (TIA).

In the US, stroke is the 5th most common cause of death and the most common cause of neurologic disability in adults. Strokes involve the arteries of the brain, either the anterior circulation (branches of the internal carotid artery) or the posterior circulation (branches of the vertebral and basilar arteries).

Ischemic stroke is sudden neurologic deficits that result from focal cerebral ischemia associated with permanent brain infarction (e.g., positive results on diffusion-weighted MRI). Common causes are (from most to least common) atherothrombotic occlusion of large arteries; cerebral embolism (embolic infarction); nonthrombotic occlusion of small, deep cerebral arteries (lacunar infarction); and proximal arterial stenosis with hypotension that decreases cerebral blood flow in arterial watershed zones (hemodynamic stroke). Diagnosis is clinical, but CT or MRI is done to exclude hemorrhage and confirm the presence and extent of stroke. Thrombolytic therapy may be useful acutely in certain patients. Depending on the cause of stroke, carotid endarterectomy or stenting, antiplatelet drugs, or warfarin may help reduce risk of subsequent strokes.

Inadequate blood flow in a single brain artery can often be compensated for by an efficient collateral system, particularly between the carotid and vertebral arteries via anastomoses at the circle of Willis and, to a lesser extent, between major arteries supplying the cerebral hemispheres. However, normal variations in the circle of Willis and in the caliber of various collateral vessels, atherosclerosis, and other acquired arterial lesions can interfere with collateral flow, increasing the chance that blockage of one artery will cause brain ischemia.

Some neurons die when perfusion is <5% of normal for >5 minutes; however, the extent of damage depends on the severity of ischemia. If it is mild, damage proceeds slowly; thus, even if perfusion is 40% of normal, 3 to 6 hours may elapse before brain tissue is completely lost. However, if severe ischemia persists >15 to 30 minutes, all of the affected tissue dies (infarction). Damage occurs more rapidly during hyperthermia and more slowly during hypothermia. If tissues are ischemic but not yet irreversibly damaged, promptly restoring blood flow may reduce or reverse injury. For example, intervention may be able to salvage the moderately ischemic areas (penumbras) that often surround areas of severe ischemia; penumbras exist because of collateral flow.

Mechanisms of ischemic injury include edema, microvascular thrombosis, programmed cell death (apoptosis), infarction with cell necrosis. Inflammatory mediators (e.g., interleukin-1B, tumor necrosis factor-alpha) contribute to edema and microvascular thrombosis. Edema, if severe or extensive, can increase intracranial pressure.

Many factors may contribute to necrotic cell death; they include loss of adenosine triphosphate (ATP) stores, loss of ionic homeostasis (including intracellular calcium accumulation), lipid peroxidative damage to cell membranes by free radicals (an iron-mediated process), excitatory neurotoxins (eg, glutamate), and intracellular acidosis due to accumulation of lactate.

Intracerebral hemorrhage usually results from rupture of an arteriosclerotic small artery that has been weakened, primarily by chronic arterial hypertension. Such hemorrhages are usually large, single, and catastrophic. Other modifiable risk factors that contribute to arteriosclerotic hypertensive intracerebral hemorrhages include cigarette smoking, obesity, and a high-risk diet (e.g., high in saturated fats, trans fats, and calories). Use of cocaine or, occasionally, other sympathomimetic drugs can cause transient severe hypertension leading to hemorrhage.

Less often, intracerebral hemorrhage results from congenital aneurysm, arteriovenous malformations, other vascular malformations, trauma, mycotic aneurysm, brain infarct (hemorrhagic infarction), primary or metastatic brain tumor, excessive anticoagulation, blood dyscrasia, intracranial arterial dissection, moyamoya disease, or a bleeding or vasculitic disorder.

Lobar intracerebral hemorrhages (hematomas in the cerebral lobes, outside the basal ganglia) usually result from angiopathy due to amyloid deposition in cerebral arteries (cerebral amyloid angiopathy), which affects primarily older people. Lobar hemorrhages may be multiple and recurrent.

Blood from an intracerebral hemorrhage accumulates as a mass that can dissect through and compress adjacent brain tissues, causing neuronal dysfunction. Large hematomas increase intracranial pressure. Pressure from supratentorial hematomas and the accompanying edema may cause transtentorial brain herniation, compressing the brain stem and often causing secondary hemorrhages in the midbrain and pons.

If the hemorrhage ruptures into the ventricular system (intraventricular hemorrhage), blood may cause acute hydrocephalus. Cerebellar hematomas can expand to block the 4th ventricle, also causing acute hydrocephalus, or they can dissect into the brain stem. Cerebellar hematomas that are >3 cm in diameter may cause midline shift or herniation.

Herniation, midbrain or pontine hemorrhage, intraventricular hemorrhage, acute hydrocephalus, or dissection into the brain stem can impair consciousness and cause coma and death.

Subarachnoid hemorrhage is sudden bleeding into the subarachnoid space. The most common cause of spontaneous bleeding is a ruptured aneurysm. Symptoms include sudden, severe headache, usually with loss or impairment of consciousness. Secondary vasospasm (causing focal brain ischemia), meningismus, and hydrocephalus (causing persistent headache and obtundation) are common. Diagnosis is by CT or MRI; if neuroimaging is normal, diagnosis is by cerebrospinal fluid analysis. Treatment is with supportive measures and neurosurgery or endovascular measures, preferably in a comprehensive stroke center.

Blood in the subarachnoid space causes a chemical meningitis that commonly increases intracranial pressure for days or a few weeks. Secondary vasospasm may cause focal brain ischemia; about 25% of patients develop signs of a transient ischemic attack (TIA) or ischemic stroke. Brain edema is maximal and risk of vasospasm and subsequent infarction (called angry brain) is highest between 72 hours and 10 days. Secondary acute hydrocephalus is also common. A 2nd rupture (rebleeding) sometimes occurs, most often within about 7 days.

Treatment for hemorrhagic transformation following endovascular treatment is similar to treatment principles for spontaneous intracerebral hemorrhage. In general, treatment involves cardiovascular and respiratory support when needed, blood pressure management, monitoring for neurological deterioration, prevention of hematoma expansion, and treatment of elevated intracranial pressure and other complications that arise from the hemorrhage including seizures. More specific treatments are well known in the art, for example, as discussed by Hemphill et al. (Stroke. 2015 July; 46 (7): 2032-60) which is specifically incorporated by reference herein.

The present invention incorporates information disclosed in other applications and texts. The following patent and other publications are hereby incorporated by reference in their entireties: Alberts et al., The Molecular Biology of the Cell, 4th Ed., Garland Science, 2002; Vogelstein and Kinzler, The Genetic Basis of Human Cancer, 2d Ed., McGraw Hill, 2002; Michael, Biochemical Pathways, John Wiley and Sons, 1999; Weinberg, The Biology of Cancer, 2007; Immunobiology, Janeway et al. 7th Ed., Garland, and Leroith and Bondy, Growth Factors and Cytokines in Health and Disease, A Multi Volume Treatise, Volumes 1A and IB, Growth Factors, 1996.

Unless otherwise apparent from the context, all elements, steps or features of the invention can be used in any combination with other elements, steps or features.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

The invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. Due to biological functional equivalency considerations, changes can be made in protein structure without affecting the biological action in kind or amount. All such modifications are intended to be included within the scope of the appended claims.

The subject methods are used for prophylactic or therapeutic purposes. As used herein, the term “treating” is used to refer to both prevention of relapses, and treatment of pre-existing conditions. For example, the prevention of inflammatory disease can be accomplished by administration of the agent prior to development of a relapse. The treatment of ongoing disease, where the treatment stabilizes or improves the clinical symptoms of the patient, is of particular interest.

All publications, patents, and patent applications mentioned in this specification are herein incorporated by reference to the same extent as if each individual publication, patent, or patent application was specifically and individually indicated to be incorporated by reference.

### Methods of the Invention

Provided herein are methods for assessing the propensity to develop hemorrhagic transformation (HT) for an individual suffering from an acute ischemic stroke following an endovascular treatment (EVT). In some embodiments, the methods comprise obtaining a cellular biological sample for analysis comprising immune cells from a patient after having the acute ischemic stroke, measuring single-cell levels of activated signaling proteins in immune cell subset(s) present in the immune cells; determining whether levels of activated signaling proteins associated with the propensity to develop hemorrhagic transformation are present; and providing an assessment of the patient's prognosis for propensity to develop hemorrhagic transformation after the endovascular treatment.

The endovascular treatment may be any treatment deemed useful in treating the acute ischemic stroke. Endovascular treatments are well known in the art and have been described in, for example, Pierot et al. (Rev Neurol (Paris). 2017 November; 173 (9): 594-599) which is specifically incorporated by reference herein. In some embodiments, the endovascular treatment is a thrombectomy. In some embodiments, the endovascular treatment is thrombolysis. In some embodiments, the thrombolysis is systemic thrombolysis. In some embodiments, the systemic thrombolysis comprises administering an effective amount of a tissue-type plasminogen activator or a urokinase-type plasminogen activator intravenously. In some embodiments, the thrombolysis is local thrombolysis. In some embodiments, the local thrombolysis comprises administering an effective amount of a tissue-type plasminogen activator or a urokinase-type plasminogen activator intra-arterially. In some embodiments, the tissue plasminogen activator is selected from the group consisting of Altepase, Reteplase, and Tenecteplase. In some embodiments, the urokinase-type plasminogen activator is urokinase.

The cellular biological sample can be any suitable type that allows for the analysis of one or more cells, preferably a blood sample. Samples can be obtained once or multiple times from an individual. Multiple samples can be obtained from different locations in the individual (e.g., blood samples, bone marrow samples, and/or lymph node samples), at different times from the individual, or any combination thereof. In some embodiments, the blood sample is a veinous blood sample or an arterial blood sample. In some embodiments, the blood sample is a veinous blood sample or arterial blood sample near the site of the acute ischemic stroke (i.e. near a thrombus).

When samples are obtained as a series, e.g., a series of blood samples obtained following an acute ischemic stroke, the samples can be obtained at fixed intervals, at intervals determined by the status of the most recent sample or samples or by other characteristics of the individual, or some combination thereof. It will be appreciated that an interval may not be exact, according to an individual's availability for sampling and the availability of sampling facilities, thus approximate intervals corresponding to an intended interval scheme are encompassed by the invention. Generally, the most easily obtained samples are fluid samples, e.g., blood samples. In some embodiments, the sample or samples are blood.

Samples may be obtained at one or more time points. Where a sample at a single time point is used, comparison is made to a reference “base line” level for the presence of the activated form of the signaling protein of interest, which may be obtained from a normal control, a pre-determined level obtained from one or a population of individuals, from a negative control for ex vivo activation, and the like.

The obtaining of the cellular biological sample may be performed at any time following the acute ischemic stroke. In some embodiments, the obtaining is performed prior to the endovascular treatment. In some embodiments, the obtaining is performed after the endovascular treatment. In some embodiments, the obtaining is performed before and after the endovascular treatment. When the obtaining is performed before and after the endovascular treatment, the cellular biological samples are measured and the results of the measuring before the endovascular treatment may be compared to the results after the endovascular treatment where the results of the measuring performed on the cellular biological sample collected before the endovascular treatment is used as a reference for the measuring performed on the cellular biological samples collected after the therapy. The comparison may be used to determine if there is an increase or decrease in the level of signaling protein activation or in the frequency of one or more specific immune cell subset(s). When the obtaining is performed before the endovascular treatment, the cellular biological sample is measured and the results of the measuring may be compared to a reference scale from a cohort of patients with known outcomes, e.g., patients who had hemorrhagic transformation following endovascular treatment and patients who did not have hemorrhagic transformation following endovascular treatment.

One or more cells or cell types, or samples containing one or more cells or cell types, can be isolated from body samples. The cells can be separated from body samples by red cell lysis, centrifugation, elutriation, density gradient separation, apheresis, affinity selection, panning, FACS, centrifugation with Hypaque, solid supports (magnetic beads, beads in columns, or other surfaces) with attached antibodies, etc. By using antibodies specific for markers identified with particular cell types, a relatively homogeneous population of cells can be obtained. Alternatively, a heterogeneous cell population can be used, e.g. circulating peripheral blood mononuclear cells.

Multiparametric analysis, at a single cell level, of cellular biological samples obtained from an individual following an acute ischemic stroke is used to obtain a determination of changes in immune cell subsets, which changes include, without limitation, altered activation states of proteins involved in signaling pathways. It is surprisingly found that changes occur in signaling pathways of these immune cells that are predictive of the propensity to develop hemorrhagic transformation (HT) following endovascular treatment (EVT) in individuals suffering from an acute ischemic stroke. For example, multiparameter flow cytometry at the single cell level measures the activation status of multiple intracellular signaling proteins, as well as assigning activation states of these proteins to the varied cell sub-sets within a complex cell population. Flow cytometry includes, without limitations FACS, mass cytometry, and the like.

Protein phosphorylation is a critical post-translational process in controlling many cell functions such as migration, apoptosis, proliferation, and differentiation. Site-specific phosphorylation of proteins can be detected, for example, by incubating cells with labeled phospho-specific antibodies prior to analysis using flow cytometry, e.g., FACS or mass cytometry. The phospho-specific antibody may comprise a label for detection when analyzed by flow cytometry. In some embodiments, the label is a fluorescent label. In some embodiments, the label is a metal isotope. The metal isotope may be any metal isotopes deemed useful including, without limitation, ⁸⁹Y, ¹¹³In, ¹¹⁵ In, ¹³⁹La, ¹⁴⁰Ce, ¹⁴¹Pr, ¹⁴²Nd, ¹⁴³Nd, ¹⁴⁴Nd, ¹⁴⁵Nd, ¹⁴⁶Nd, ¹⁴⁷Sm, ¹⁴⁸Nd, ¹⁵²Sm, ¹⁵⁶Gd, ¹⁵⁷Gd, ¹⁵⁸Gd, ¹⁶³Dy, ¹⁶⁵Ho, ¹⁶⁹Tm, ¹⁷⁰Er, ¹⁷¹Yb, ¹⁷²Yb, ¹⁷³Yb, ¹⁷⁴Yb, ¹⁷⁵Yb, ¹⁷⁶Yb, ²⁰⁹Bi, ¹¹³In, ¹⁴⁹Sm, ¹⁵⁰Nd, ¹⁵¹ Eu, ¹⁵³Eu, ¹⁵⁴Sm, ¹⁵⁵Gd, ¹⁵⁹Tb, ¹⁶⁰Gd, ¹⁶¹ Dy, ¹⁶²Dy, ¹⁶⁴Dy, ¹⁶⁶Er, ¹⁶⁷Er, ¹⁶⁸Er, etc.

The methods of the present disclosure involve measuring single-cell levels of activated signaling proteins in immune cell subset(s). The measuring may comprise any non-invasive method of quantifying activated signaling proteins in the immune cell subset(s) in the cellular biological sample. In general, the measuring comprises physically contacting cells with a panel of affinity reagents specific for activated signaling proteins and for markers that distinguish subsets of immune cells. Usually, the affinity reagents comprise a detectable label, e.g., metal isotope, fluorophore, etc. The signal intensity of the markers is measured, preferably at a single-cell level. Suitable methods of measuring include, without limitation, flow cytometry, mass cytometry, confocal microscopy, and the like. The data, which can include measurements of the intensity of signaling molecules and phosphorylation status in selected immune cell subsets, etc., is compared to measurements of the same from the baseline cell population. The data can be normalized for comparison.

In some embodiments of the invention, different gating strategies can be used in order to analyze a specific cell population (e.g., only CD4+ T cells or Th1 CD4+ T cells) in a sample of mixed cell population. These gating strategies can be based on the presence of one or more specific surface markers. Gating strategies for sorting CD4+ T cells are well known in the art and have been described in, for example, Mousset et al. (Cytometry A. 2019 June; 95 (6): 647-654) which is specifically incorporated by reference herein. In some embodiments, the measuring is also gated on neutrophils. A clear comparison can be carried out by using two-dimensional contour plot representations, two-dimensional dot plot representations, and/or histograms.

The immune cells are analyzed for the presence of an activated form of a signaling protein of interest. Signaling proteins of interest include, without limitation, pSTAT1, 3, 5, 6, pERK, p38, pMK2, prpS6, pCREB, pNF-_{κ}B, total I_{κ}B, prpS6 and pCREB are of particular interest. The changes in signal protein activation are identified using specific reagents that are able to differentiate between activated and non-activated states (e.g. an antibody). In some embodiments, signaling proteins involved in the mTOR pathway are of interest. Non-limiting examples of signaling proteins involved in the mTOR pathway are PDCD4, rpS6, eEF2L elF4B, mdm2, BAD, CREMt, SKAR, Rictor, IRS-1, mTOR, S6K, and 4E-BP1. Other signal proteins involved in the mTOR pathway are well known in the art and have been described in, for example, Showkat et al. (Mol Biol Int. 2014; 2014:686984) which is specifically incorporated by reference herein. To determine if a change is significant the signal in a patient's baseline sample can be compared to a reference scale from a cohort of patients with known outcomes.

The differential presence of these markers is shown to provide for prognostic evaluations to detect individuals having a propensity to develop HT following EVT. In general, such prognostic methods involve determining the presence or level of activated signaling proteins in an individual sample of immune cells. Detection can utilize one or a panel of specific binding members, e.g. a panel or cocktail of binding members specific for one, two, three, four, five, or more markers.

In some embodiments, a phenotypic profile of a population of cells is determined by measuring the activation level of a signaling protein. The methods of the invention can be employed to examine and profile the status of any signaling protein in a cellular pathway, or collections of such signaling proteins. Single or multiple distinct pathways can be profiled (sequentially or simultaneously, such as the mTOR pathway), or subsets of signaling proteins within a single pathway or across multiple pathways can be examined (sequentially or simultaneously).

In some embodiments, the basis for classifying cells is that the distribution of activation levels for one or more specific signaling proteins will differ among different phenotypes. A certain activation level, or more typically a range of activation levels for one or more signaling proteins seen in a cell or a population of cells, is indicative that that cell or population of cells belongs to a distinctive phenotype. Other measurements, such as cellular levels (e.g., expression levels) of biomolecules that may not contain signaling proteins, can also be used to classify cells in addition to activation levels of signaling proteins; it will be appreciated that these levels also will follow a distribution. Thus, the activation level or levels of one or more signaling proteins, optionally in conjunction with the level of one or more biomolecules that may or may not contain signaling proteins, of a cell or a population of cells can be used to classify a cell or a population of cells into a class. It is understood that activation levels can exist as a distribution and that an activation level of a particular element used to classify a cell can be a particular point on the distribution but more typically can be a portion of the distribution. In addition to activation levels of intracellular signaling proteins, levels of intracellular or extracellular biomolecules, e.g., proteins, can be used alone or in combination with activation states of signaling proteins to classify cells. Further, additional cellular elements, e.g., biomolecules or molecular complexes such as RNA, DNA, carbohydrates, metabolites, and the like, can be used in conjunction with activation states or expression levels in the classification of cells encompassed here.

When necessary, cells are dispersed into a single-cell suspension, e.g. by enzymatic digestion with a suitable protease, e.g. collagenase, dispase, etc; and the like. An appropriate solution is used for dispersion or suspension. Such solution will generally be a balanced salt solution, e.g. normal saline, PBS, Hanks balanced salt solution, etc., conveniently supplemented with fetal calf serum or other naturally occurring factors, in conjunction with an acceptable buffer at low concentration, generally from 5-25 mM. Convenient buffers include HEPES1 phosphate buffers, lactate buffers, etc. The cells can be fixed, e.g. with 3% paraformaldehyde, and are usually permeabilized, e.g. with ice cold methanol; HEPES-buffered PBS containing 0.1% saponin, 3% BSA; covering for 2 min in acetone at −200 C; and the like as known in the art and according to the methods described herein.

The signaling proteins may be activated in all immune cell subsets or one or more specific immune cell subsets. Immune cell subsets of interest include both innate and adaptive immune cell subsets. Immune cell subsets that find use in the present disclosure include without limitation, CCR2-nonclassical monocytes, CCR2+ classical monocytes, CD56brightCD16-natural killer (NK) cells, CD56dimCD16+ NK cells, CD62L− aged neutrophils, CD62L+ immature neutrophils, CD66+ neutrophils, intermediate monocytes, myeloid dendritic cells, plasmacytoid dendritic cells, myeloid-derived suppressor cells, CD4+ central memory T cells, CD4+ effector memory T cells, CD4+ naive T cells, CD4+ resident memory T cells, CD8+ central memory T cells, CD8+ effector memory T cells, CD8+ naive T cells, CD8+ resident memory T cells, gamma-delta T cells, natural-killer T cells, memory T helper 1 (Th1) cells, naive Th1 cells, memory regulatory T cells, and naive regulatory T cells, etc. In some embodiment, increased prpS6 signal protein activation in memory Th1 CD4+ T cells and neutrophils is indicative of an increased likelihood for an individual to have a HT following EVT. In some embodiment, increased prpS6 signal protein activation in CD66+ neutrophils is indicative of an increased likelihood for an individual to have a HT following EVT. In some embodiment, increased prpS6 signal protein activation in B cells is indicative of increased likelihood for an individual to have a HT following EVT. In some embodiment, increased pCREB signal protein activation in naive Th1 CD4+ T cells is indicative of an increased likelihood for an individual to have a HT following EVT. In some embodiment, increased pNF-κB signal protein activation in non-classical monocytes is indicative of an increased likelihood for an individual to have a HT following EVT. In some embodiment, increased pMAPKAPK2 signal protein activation in non-classical monocytes is indicative of an increased likelihood for an individual to have a HT following EVT.

In addition to measuring the single-cell levels of activated signaling proteins, the methods may further comprise measuring the frequency of specific immune cell subtypes may also be indicative of an individual's likelihood or propensity to develop HT following EVT. The frequency of a specific immune cell subtype may be high or low relative to a reference state or as determined by a qualified professional (i.e. a physician). In some embodiments, a high or increased frequency of non-classical monocytes is indicative of increased likelihood for an individual to develop HT following EVT. In some embodiments, the combination of a high or increased prpS6 signal protein activation in CD66+ neutrophils, memory Th1 CD4+ T cells and B cells, increased pCREB signal protein activation in naive Th1 CD4+ T cells, increased pNF-_{κ}B and pMAPKAPK2 signal protein activation in non classical monocyte, and a high or increased frequency of non-classical monocytes and classical monocytes is indicative of increased likelihood for an individual to have a HT following EVT.

An aspect of the method is determining whether changes in the levels of activated signaling proteins associated with the propensity to develop HT are present. The determining may be performed by integrating the results of the measuring step into a multivariate model. The level of one or more activated signaling proteins from one or more of the immune cell subset(s) may be integrated into the multivariate model. The level of one or more activated signaling proteins includes two or more, three or more, four or more, five or more, six or more, seven or more, either or more, nine or more, ten or more, eleven or more, twelve or more, or the level thirteen or more activated signaling proteins. The level of one or more activated signal proteins from one or more of any of the immune subset(s) described above may be integrated into the multivariate model and a score is outputted that is indicative of whether or not the individual is at increased risk of having HT following EVT. The one or more the immune subset(s) includes two or more, three or more, four or more, five or more, six or more, seven or more, either or more, nine or more, ten or more, eleven or more, twelve or more, thirteen or more, fourteen or more, fifteen or more, sixteen or more, seventeen or more, eighteen or more, nineteen or more, twenty or more, twenty one or more, twenty two or more, twenty three or more, twenty four or more, or twenty five or more immune cell subset(s).

In some embodiments, the level of prpS6 in memory Th1 CD4+ T cells is integrated into the multivariate model and a score is outputted that is indicative of whether or not the individual is at increased risk of having HT following EVT. In some embodiments, the level of prpS6 in CD66+ neutrophils cells is integrated into the multivariate model and a score is outputted that is indicative of whether or not the individual is at increased risk of having HT following EVT. In some embodiments, the level of prpS6 in B cells is integrated into the multivariate model and a score is outputted that is indicative of whether or not the individual is at increased risk of having HT following EVT. In some embodiments, the level of pCREB in naive Th1 CD4+ T cells is integrated into the multivariate model and a score is outputted that is indicative of whether or not the individual is at increased risk of having HT following EVT. In some embodiments, the frequency of non-classical monocytes is integrated into the multivariate model and a score is outputted that is indicative of whether or not the individual is at increased risk of having HT following EVT. In some embodiments, the frequency of classical monocytes is integrated into the multivariate model and a score is outputted that is indicative of whether or not the individual is at increased risk of having HT following EVT. In some embodiments, the level of pNF-κB in non-classical monocytes is integrated into the multivariate model and a score is outputted that is indicative of whether or not the individual is at increased risk of having HT following EVT. In some embodiments, the level of pMAPKAPK2 in non-classical monocytes is integrated into the multivariate model and a score is outputted that is indicative of whether or not the individual is at increased risk of having HT following EVT. In some embodiments, any combination of the above activated signal proteins in any combination of the above immune cell subset(s) is integrated into the multivariate model and a score is outputted that is indicative of whether or not the individual is at increased risk of having HT following EVT. In some embodiments, the level of prpS6 in memory Th1 CD4+ T cells, the level of pCREB in naive Th1 CD4+ T cells, and the frequency of non-classical monocytes are integrated into the multivariate model and a score is outputted that is indicative of whether or not the individual is at high increased of having HT following EVT. In some embodiments, the level of prpS6 in CD66+ neutrophils, memory Th1 CD4+ T cells and B cells, the level of pCREB in naive Th1 CD4+ T cells, the level of pNF-κB and pMAPKAPK2 in non-classical monocytes, and the frequency of classical monocytes and non-classical monocytes are integrated into the multivariate model and a score is outputted that is indicative of whether or not the individual is at high increased of having HT following EVT.

Individuals showing immune features indicative of HT can be treated accordingly. Because the diagnosis can be provided significantly before clinical symptoms or the occurrence of HT, the methods herein provide a means of timely intervention. In addition, the results provided by the methods with assist in the determination of the timing of the endovascular treatment, the use of immune-modifying therapies, e.g., mTOR inhibitors, in addition to the EVT in order to prevent the occurrence of the HT following a determination of increased likelihood of HT following EVT, and will assist in the determination of timing and disposition of discharge from the hospital and clinical follow up.

In some embodiments, when the individual is determined to have a propensity to develop HT following EVT, the EVT may be delayed by 1-24 hours. For instance, the EVT may be delayed by 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours. In some embodiments, if the individual is determined to have a propensity to develop HT following EVT, an EVT is not performed.

In some embodiments, when the individual is determined to have a propensity to develop HT following EVT, the obtaining of the cellular biological sample may be repeated 1-24 hours after the individual was determined to have a propensity to develop HT following EVT. The measuring may then be repeated following the repeated obtaining of the cellular biological sample to determine if the individual has a continued propensity to develop HT following EVT.

In some embodiments, when the individual is determined to have a propensity to develop HT following EVT, before, during, or after the EVT an mTOR inhibitor is administered. The mTOR inhibitor may be any mTOR inhibitor deemed useful including, without limitation, sirolimus (Rapamycin), everolimus, temsirolimus, deforolimus (also known as ridaforolimus), umirolimus, zotarolimus, rapalogs (rapamycin analogs), etc. In some embodiments, when the individual is determined to have a propensity to develop HT following EVT, before the EVT an mTOR inhibitor is administered. In some embodiments, when the individual is determined to have a propensity to develop HT following EVT, during the EVT an mTOR inhibitor is administered. In some embodiments, when the individual is determined to have a propensity to develop HT following EVT, after the EVT an mTOR inhibitor is administered.

In some embodiments, if the individual is determined not to have a propensity to develop HT following EVT, the EVT is performed immediately. In some embodiments, if the individual is determined not to have a propensity to develop HT following EVT, the EVT is performed within 1-6 hours of the determination. The EVT may be any EVT deemed appropriate. In some embodiments, the EVT is thrombolysis. In some embodiments, the thrombolysis is systemic thrombolysis. In some embodiments, the systemic thrombolysis comprises administering an effective amount of a tissue-type plasminogen activator or a urokinase-type plasminogen activator intravenously. In some embodiments, the thrombolysis is local thrombolysis. In some embodiments, the local thrombolysis comprises administering an effective amount of a tissue-type plasminogen activator or a urokinase-type plasminogen activator intra-arterially. In some embodiments, the tissue plasminogen activator is selected from the group consisting of altepase, reteplase, and tenecteplase. In some embodiments, the urokinase-type plasminogen activator is urokinase.

Also described herein is a method for assessing prognosis for HT following EVT, comprising: obtaining a dataset associated with a sample obtained from the individual suffering from the acute ischemic stroke, wherein the dataset comprises quantitative data for specific immune cell subset and the activated signaling proteins contained therein; and analyzing the dataset for changes at the single cell level for these markers, wherein a statistically significant match with a HT pattern is indicative of the prognosis to develop HT following EVT. The data may be analyzed by a computer processor. The processor may be communicatively coupled to a storage memory for analyzing the data. Also described herein is a computer-readable storage medium storing computer-executable program code, the program code comprising: program code for storing and analyzing data obtained by the methods of the invention.

### Data Analysis

A signature pattern can be generated from a biological sample using any convenient protocol, for example as described below. The readout can be a mean, average, median or the variance or other statistically or mathematically-derived value associated with the measurement. The marker readout information can be further refined by direct comparison with the corresponding reference or control pattern. A binding pattern can be evaluated on a number of points: to determine if there is a statistically significant change at any point in the data matrix relative to a reference value; whether the change is an increase or decrease in the binding; whether the change is specific for one or more physiological states, and the like. The absolute values obtained for each marker under identical conditions will display a variability that is inherent in live biological systems and also reflects the variability inherent between individuals.

Following obtainment of the signature pattern from the sample being assayed, the signature pattern can be compared with a reference or base line profile to make a prognosis regarding the phenotype of the patient from which the sample was obtained/derived. Additionally, a reference or control signature pattern can be a signature pattern that is obtained from a sample of a patient known to not have had a hemorrhagic transformation following endovascular treatment.

In certain embodiments, the obtained signature pattern is compared to a single reference/control profile to obtain information regarding the phenotype of the patient being assayed. In yet other embodiments, the obtained signature pattern is compared to two or more different reference/control profiles to obtain more in depth information regarding the phenotype of the patient. For example, the obtained signature pattern can be compared to a positive and negative reference profile to obtain confirmed information regarding whether the patient has the phenotype of interest.

Samples can be obtained from the tissues or fluids of an individual. For example, samples can be obtained from whole blood, tissue biopsy, serum, etc. Other sources of samples are body fluids such as lymph, cerebrospinal fluid, and the like. Also included in the term are derivatives and fractions of such cells and fluids.

In order to identify profiles that are indicative of responsiveness, a statistical test can provide a confidence level for a change in the level of markers between the test and reference profiles to be considered significant. The raw data can be initially analyzed by measuring the values for each marker, usually in duplicate, triplicate, quadruplicate or in 5-10 replicate features per marker. A test dataset is considered to be different than a reference dataset if one or more of the parameter values of the profile exceeds the limits that correspond to a predefined level of significance.

The analysis of immune features such as the level of prpS6 in memory Th1 CD4+ T cells, the level of pCREB in naive Th1 CD4+ T cells, and the frequency of non-classical monocytes are integrated into the multivariate model and a score is outputted that is indicative of whether or not the individual is at increased risk of having HT following EVT. The analysis of immune features such as the level of prpS6 in CD66+ neutrophils, memory Th1 CD4+ T cells, and B cells, the level of pCREB in naive Th1 CD4+ T cells, the level of pNF-κB and pMAPKAPK2 in non-classical monocytes and the frequency of non-classical monocytes and classical monocytes are integrated into the multivariate model and a score is outputted that is indicative of whether or not the individual is at increased risk of having HT following EVT. The multivariate model was produced using a sparse machine learning algorithm. The sparse machine learning algorithm was trained on the analysis of 25 immune cell subsets with 47 different parameters. The 25 different immune subsets analyzed included CCR2-nonclassical monocytes, CCR2+ classical monocytes, CD56brightCD16-natural killer (NK) cells, CD56dimCD16+ NK cells, CD62L− aged neutrophils, CD62L+ immature neutrophils, CD66+ neutrophils, intermediate monocytes, myeloid dendritic cells, plasmacytoid dendritic cells, myeloid-derived suppressor cells, CD4+ central memory T cells, CD4+ effector memory T cells, CD4+ naive T cells, CD4+ resident memory T cells, CD8+ central memory T cells, CD8+ effector memory T cells, CD8+ naive T cells, CD8+ resident memory T cells, gamma-delta T cells, natural-killer T cells, memory T helper 1 (Th1) cells, naive Th1 cells, memory regulatory T cells, and naive regulatory T cells. The parameters analyzed in identifying important immune features included, without limitation, CD41, CD235ab, CD45, CD66, KIR, CD7, CD19, CD45RA, CD11b, CD4, CD8a, CD11c, CD123, TCRγδ, FceRIα, CD161, CD33, CRTH2, CD16, CD25, CD3, CXCR4, CD62L, CCR2, HLA-DR, CD14, CD56, CD61, cPARP, pCREB, pSTAT5, pp38, pSTAT1, pSTAT3, prpS6, pMAPKAPK2, Tbet, Ki67, FoxP3, IκB, pNFκB, pERK1/2, pSTAT6, etc.

The sparse machine learning algorithm employed STABL, a machine learning framework that unifies the biomarker discovery process with multivariate predictive modeling of clinical outcomes by choosing a selective and reliable set of biomarkers. The STABL algorithm has been disclosed in, for example, International Patent Application WO2022/198239 and Hedou et al. (Res Sq.2023 Feb. 28: rs.3.rs-2609859) each specifically incorporated by reference herein. The STABL algorithm functions as follows: Starting with a matrix of n observations with p features (informative and uninformative), no-variance features are removed, and the data is z-scored. Noise is then injected into the original dataset by creating p artificial features via random permutations or Knockoff sampling from the original data matrix (p vectors of size n).

The two feature sets are concatenated to form a new matrix of n observations with 2p features. A bootstrap procedure is then performed on this noise-injected matrix: at each bootstrap repetition, a set of LASSO (or other sparse algorithm) models are fit over a range of regularization parameters Λ. For each model at regularization parameter λ∈Λ, features with non-zero βs are selected.

Following the stability selection methodology, the selected features are tracked in each model, and the frequency of selection

$${π}_{i}^{λ}$$

of each feature i (original or artificial) for each value of λ is computed. The distribution of

$${π}_{i} = {max}_{λ} {π}_{i}^{λ}$$

is used to rank the features and to construct a reliability threshold.

Let S be the set of original features (informative and uninformative) and N the set of artificial features, which are uninformative by construction. The reliability threshold p is defined as the value of possible frequency thresholds (t) that minimizes the FDRc estimate:

$$ρ = arg {min}_{t} {\text{?}}_{c} ( t ) = arg {min}_{t} \frac{\# \{ {π}_{i} > t \vert i ∈ N \} + 1}{\# \{ {π}_{i} > t \vert i ∈ N \} + \# \{ {π}_{i} > t \vert i ∈ S \}}$$

Only features for which πᵢ>ρ are selected in the final set of features, hereafter referred to as stable features. Thus defined, ρ limits the proportion of false positives in the final model, ensuring the reliability of the selected feature set.

In the case of multi-omics integration, only stable features selected from each omics are used and combined into a final model for the prediction of the clinical endpoint. The input of this final model is a matrix of n observations by p_{stable} selected features. p_{stable} is usually significantly lower than the original number of selected features p. Selective features can integrated into non-sparse algorithms, such as regressions, for the final model.

The determining in the methods described above may be performed by integrating the results of the measuring step into a multivariate model produced by the sparse machine learning algorithm described above. In some embodiments, the level of prpS6 in memory Th1 CD4+ T cells is integrated into the multivariate model and a score is outputted that is indicative of whether or not the individual is at increased risk of having HT following EVT. In some embodiments, the level of prpS6 in CD66+ neutrophils cells is integrated into the multivariate model and a score is outputted that is indicative of whether or not the individual is at increased risk of having HT following EVT. In some embodiments, the level of prpS6 in B cells is integrated into the multivariate model and a score is outputted that is indicative of whether or not the individual is at increased risk of having HT following EVT. In some embodiments, the level of pCREB in naive Th1 CD4+ T cells is integrated into the multivariate model and a score is outputted that is indicative of whether or not the individual is at increased risk of having HT following EVT. In some embodiments, the frequency of non-classical monocytes is integrated into the multivariate model and a score is outputted that is indicative of whether or not the individual is at increased risk of having HT following EVT. In some embodiments, the frequency of classical monocytes is integrated into the multivariate model and a score is outputted that is indicative of whether or not the individual is at increased risk of having HT following EVT. In some embodiments, the level of pNF-κB in non-classical monocytes is integrated into the multivariate model and a score is outputted that is indicative of whether or not the individual is at increased risk of having HT following EVT. In some embodiments, the level of pMAPKAPK2 in non-classical monocytes is integrated into the multivariate model and a score is outputted that is indicative of whether or not the individual is at increased risk of having HT following EVT.

In some embodiments, any combination of the above activated signal proteins in any combination of the above immune cell subset(s) is integrated into the multivariate model and a score is outputted that is indicative of whether or not the individual is at increased risk of having HT following EVT. In some embodiments, the level of prpS6 in memory Th1 CD4+ T cells, the level of pCREB in naive Th1 CD4+ T cells, and the frequency of non-classical monocytes are integrated into the multivariate model and a score is outputted that is indicative of whether or not the individual is at high increased of having HT following EVT. In some embodiments, the level of prpS6 in CD66+ neutrophils, memory Th1 CD4+ T cells and B cells, the level of pCREB in naive Th1 CD4+ T cells, the level of pNF-κB and pMAPKAPK2 in non-classical monocytes, and the frequency of classical monocytes and non-classical monocytes are integrated into the multivariate model and a score is outputted that is indicative of whether or not the individual is at high increased of having HT following EVT.

### Reports

In some embodiments, providing an evaluation of a subject for a classification, diagnosis, prognosis, theranosis, and/or prediction of an outcome following endovascular treatment in an individual suffering from an acute ischemic stroke includes generating a written report that includes the artisan's assessment of the subject's state of health i.e. a “diagnosis assessment”, of the subject's prognosis, i.e. a “prognosis assessment”, and/or of possible treatment regimens, i.e. a “treatment assessment”. Thus, a subject method may further include a step of generating or outputting a report providing the results of a diagnosis assessment, a prognosis assessment, or treatment assessment, which report can be provided in the form of an electronic medium (e.g., an electronic display on a computer monitor), or in the form of a tangible medium (e.g., a report printed on paper or other tangible medium).

A “report,” as described herein, is an electronic or tangible document which includes report elements that provide information of interest relating to a diagnosis assessment, a prognosis assessment, and/or a treatment assessment and its results. A subject report can be completely or partially electronically generated. A subject report includes at least a diagnosis assessment, i.e. a diagnosis as to whether a subject will have a particular clinical response following endovascular treatment, and/or a suggested course of treatment to be followed. A subject report can further include one or more of: 1) information regarding the testing facility; 2) service provider information; 3) subject data; 4) sample data; 5) an assessment report, which can include various information including: a) test data, where test data can include an analysis of cellular signaling responses to activation, b) reference values employed, if any.

The report may include information about the testing facility, which information is relevant to the hospital, clinic, or laboratory in which sample gathering and/or data generation was conducted. This information can include one or more details relating to, for example, the name and location of the testing facility, the identity of the lab technician who conducted the assay and/or who entered the input data, the date and time the assay was conducted and/or analyzed, the location where the sample and/or result data is stored, the lot number of the reagents (e.g., kit, etc.) used in the assay, and the like. Report fields with this information can generally be populated using information provided by the user.

The report may include information about the service provider, which may be located outside the healthcare facility at which the user is located, or within the healthcare facility. Examples of such information can include the name and location of the service provider, the name of the reviewer, and where necessary or desired the name of the individual who conducted sample gathering and/or data generation. Report fields with this information can generally be populated using data entered by the user, which can be selected from among pre-scripted selections (e.g., using a drop-down menu). Other service provider information in the report can include contact information for technical information about the result and/or about the interpretive report.

The report may include a subject data section, including subject medical history as well as administrative subject data (that is, data that are not essential to the diagnosis, prognosis, or treatment assessment) such as information to identify the subject (e.g., name, subject date of birth (DOB), gender, mailing and/or residence address, medical record number (MRN), room and/or bed number in a healthcare facility), insurance information, and the like), the name of the subject's physician or other health professional who ordered the susceptibility prediction and, if different from the ordering physician, the name of a staff physician who is responsible for the subject's care (e.g., primary care physician).

The report may include a sample data section, which may provide information about the biological sample analyzed, such as the source of biological sample obtained from the subject (e.g. blood, type of tissue, etc.), how the sample was handled (e.g. storage temperature, preparatory protocols) and the date and time collected. Report fields with this information can generally be populated using data entered by the user, some of which may be provided as pre-scripted selections (e.g., using a drop-down menu).

The report may include an assessment report section, which may include information generated after processing of the data as described herein. The interpretive report can include a prognosis of the likelihood that the patient will develop hemorrhagic transformation. The interpretive report can include, for example, results of the analysis, methods used to calculate the analysis, and interpretation, i.e. prognosis. The assessment portion of the report can optionally also include a Recommendation(s). For example, where the results indicate the subject's prognosis for propensity to develop hemorrhagic transformation.

It will also be readily appreciated that the reports can include additional elements or modified elements. For example, where electronic, the report can contain hyperlinks which point to internal or external databases which provide more detailed information about selected elements of the report. For example, the patient data element of the report can include a hyperlink to an electronic patient record, or a site for accessing such a patient record, which patient record is maintained in a confidential database. This latter embodiment may be of interest in an in-hospital system or in-clinic setting. When in electronic format, the report is recorded on a suitable physical medium, such as a computer readable medium, e.g., in a computer memory, zip drive, CD, DVD, etc.

It will be readily appreciated that the report can include all or some of the elements above, with the proviso that the report generally includes at least the elements sufficient to provide the analysis requested by the user (e.g., a diagnosis, a prognosis, or a prediction of responsiveness to a therapy).

### EXPERIMENTAL

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

Provided is a method for assessing a patient's risk of developing hemorrhagic transformation (HT) following treatment for acute ischemic stroke comprised of the following: 1) collection of a veinous or arterial blood sample in a patient suffering from an acute ischemic stroke before and/or after endovascular treatment; 2) quantification of the distribution and intracellular signaling protein activity of multiple immune cell subsets in a peripheral blood sample, using suspension mass cytometry; 3) calculation of a risk score for the prediction of hemorrhagic transformation using a machine learning algorithm integrating biological data and electronic medical record data.

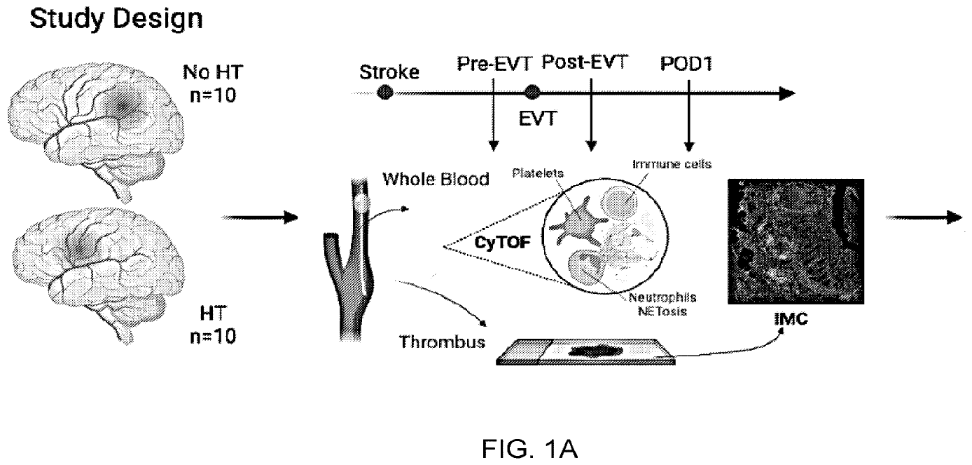

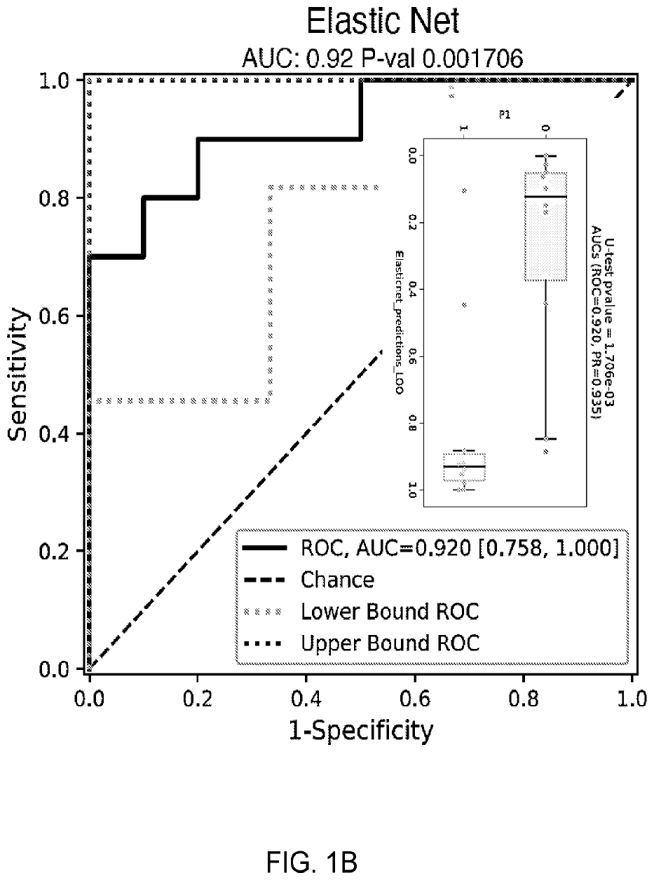

Methods: Twenty patients admitted to the hospital for endovascular treatment (EVT) after an acute ischemic stroke (AIS) were enrolled in a single-center prospective study (Table 1). The primary outcome of the study was the development of hemorrhagic transformation (HT) within 3 days after EVT. Arterial and venous whole blood samples were collected before EVT then 1 h, and 24 h after EVT. Key signaling responses implicated in the post-stroke immune response (including mTOR/LPS/TNFα, IL-1β/2/4/6) were focused on. For each sample, the frequency and intracellular signaling responses (including pSTAT1,3,5,6, pERK, p38, pMK2, prpS6, pCREB, pNF-κB, and total IκB) of 25 major innate and adaptive immune cell subset and two platelet populations (Table 2) were quantified using a custom 47-parameter mass cytometry (Table 3; 4). Multivariate predictive modeling of the primary outcome was performed using a novel sparse machine learning algorithm and predictive performance was evaluated with cross-validation (FIG. 1).

STABL was employed, a machine learning framework that unifies the biomarker discovery process with multivariate predictive modeling of clinical outcomes by choosing a selective and reliable set of biomarkers. The steps of the STABL algorithm are as follows:

1. Starting with a matrix of n observations with p features (informative and uninformative), no-variance features are removed, and the data is z-scored. Noise is then injected into the original dataset by creating p artificial features via random permutations or Knockoff sampling from the original data matrix (p vectors of size n).

2. The two feature sets are concatenated to form a new matrix of n observations with 2p features. A bootstrap procedure is then performed on this noise-injected matrix: at each bootstrap repetition, a set of LASSO (or other sparse algorithm) models are fit over a range of regularization parameters Λ. For each model at regularization parameter λ∈Λ, features with non-zero βs are selected.

3. Following the stability selection methodology, the selected features are tracked in each model, and the frequency of selection

$${π}_{i}^{λ}$$

of each feature i (original or artificial) for each value of λ is computed. The distribution of

$${π}_{i} = {max}_{λ} {π}_{i}^{λ}$$

is used to rank the features and to construct a reliability threshold.

4. Let S be the set of original features (informative and uninformative) and N the set of artificial features, which are uninformative by construction. The reliability threshold p is defined as the value of possible frequency thresholds (t) that minimizes the FDRc estimate:

$$ρ = arg {min}_{t} {\text{?}}_{c} ( t ) = arg {min}_{t} \frac{\# \{ {π}_{i} > t \vert i ∈ N \} + 1}{\# \{ {π}_{i} > t \vert i ∈ N \} + \# \{ {π}_{i} > t \vert i ∈ S \}}$$

5. Only features for which πᵢ>ρ are selected in the final set of features, hereafter referred to as stable features. Thus defined, ρ limits the proportion of false positives in the final model, ensuring the reliability of the selected feature set.

6. In the case of multi-omics integration, only stable features selected from each omics are used and combined into a final model for the prediction of the clinical endpoint. The input of this final model is a matrix of n observations by p_{stable} selected features. p_{stable} is usually significantly lower than the original number of selected features p. We can therefore integrate the selected features into non-sparse algorithms, such as regressions, for the final model.

TABLE 1
Patient demographics and outcomes

| | | | Hemorrhagic |
|---|---|---|---|
| ID | Age | Gender | transformation |
| 1377 | 86 | F | No |
| 1400 | 73 | F | No |
| 1428 | 50 | F | No |
| 1431 | 74 | F | No |
| 1326 | 90 | M | No |
| 1331 | 62 | M | No |
| 1337 | 63 | M | No |
| 1357 | 71 | M | No |
| 1418 | 88 | M | No |
| 1464 | 28 | M | No |
| 1330 | 89 | F | Yes |
| 1381 | 70 | F | Yes |
| 1472 | 67 | F | Yes |
| 1475 | 72 | F | Yes |
| 1488 | 81 | F | Yes |
| 1414 | 77 | M | Yes |
| 1427 | 53 | M | Yes |
| 1469 | 74 | M | Yes |
| 1473 | 45 | M | Yes |
| 1480 | 47 | M | Yes |

TABLE 2
Cell subsets analyzed

| Innate immune | Adaptive immune | Platelet |
|---|---|---|
| cell subset | cell subset | cell subset |
| CCR2− nonclassical monocytes | CD4+ central memory T cells | CD41high CD61high platelets |
| CCR2+ classical monocytes | CD4+ effector memory T cells | CD61+CD41+ platelets |
| CD56brightCD16− natural | CD4+ naive T cells | |
| killer (NK) cells | | |
| CD56dimCD16+ NK cells | CD4+ resident memory T cells | |
| CD62L− aged neutrophils | CD8+ central memory T cells | |
| CD62L+ immature neutrophils | CD8+ effector memory T cells | |
| intermediate monocytes | CD8+ naive T cells | |
| myeloid dendritic cells | CD8+ resident memory T cells | |
| plasmacytoid dendritic cells | gamma-delta T cells | |
| myeloid-derived | natural-killer T cells | |
| suppressor cells | | |
| | memory T helper 1 (Th1) cells | |
| | naive Th1 cells | |
| | memory regulatory T cells | |
| | naive regulatory T cells | |

TABLE 3
Mass cytometry antibody panel

| | Atomic | Atomic |
|---|---|---|
| Antigen | Symbol | Mass |
| CD41 | Y | 89 |
| CD235ab | In | 113 |
| CD45 | In | 115 |
| CD66 | La | 139 |
| KIR | Ce | 140 |
| CD7 | Pr | 141 |
| CD19 | Nd | 142 |
| CD45RA | Nd | 143 |
| CD11b | Nd | 144 |
| CD4 | Nd | 145 |
| CD8a | Nd | 146 |
| CD11c | Sm | 147 |
| CD123 | Nd | 148 |
| TCRγδ | Sm | 152 |
| FceRIα | Gd | 156 |
| CD161 | Gd | 157 |
| CD33 | Gd | 158 |
| CRTH2 | Dy | 163 |
| CD16 | Ho | 165 |
| CD25 | Tm | 169 |
| CD3 | Er | 170 |
| CXCR4 | Yb | 171 |
| CD62L | Yb | 172 |
| CCR2 | Yb | 173 |
| HLA-DR | Yb | 174 |
| CD14 | Yb | 175 |
| CD56 | Yb | 176 |
| CD61 | Bi | 209 |
| cPARP | In | 113 |
| pCREB | Sm | 149 |
| pSTAT5 | Nd | 150 |
| pp38 | Eu | 151 |
| pSTAT1 | Eu | 153 |
| pSTAT3 | Sm | 154 |
| pS6 | Gd | 155 |
| pMAPKAPK2 | Tb | 159 |
| Tbet | Go | 160 |
| Ki67 | Dy | 161 |
| FoxP3 | Dy | 162 |
| IκB | Dy | 164 |
| pNFκB | Er | 166 |
| PERK½ | Er | 167 |
| pSTAT6 | Er | 168 |

Results: A multivariate model integrating single-cell data collected pre-EVT accurately classified patients who developed HT from patients with (n=10) and without (n=10) HT. The most informative immune features of the multivariate model included markedly increased prpS6 signal in Th1 CD4+ T cells and neutrophils in patients with HT, suggesting enhanced mTOR signaling responses in multiple inflammatory cell subsets precedes the development of HT. Additional features identified in the model include frequency of ncMC and pCREB activity in Th1 CD4+ T cells.

Discussion: The pre-operative assessment of single-cell biomarkers provides a novel method for accurately predicting HT following EVT. The integrative model's predictive power was superior to existing risk assessment tools that are solely based on the assessment of clinical variables. The features identified by the integrative model can be easily measured in patients' blood using a simple venipuncture and clinically-approved mass cytometry platforms, providing a set of non-invasive biomarkers for the prediction of HT. Importantly, these predictive biomarkers pointed at biologically plausible immune mechanisms underlying the pathogenesis of HT, including dysfunctional mTOR signaling responses, and possible targets for innovative immunomodulatory treatments.

### REFERENCES

1. Mazighi M, Richard S, Lapergue B, Sibon I, Gory B, Berge J, et al. Safety and efficacy of intensive blood pressure lowering after successful endovascular therapy in acute ischaemic stroke (bp-target): A multicentre, open-label, randomised controlled trial. Lancet neurology. 2021; 20:265-274
2 Olivot J M, Heit J J, Mazighi M, Raposo N, Albucher J F, Rousseau V, et al. What predicts poor outcome after successful thrombectomy in early time window? J Neurointerv Surg. 2021-In Press
3. Gaudilliere B, Fragiadakis G K, Bruggner R V, Nicolau M, Finck R, Tingle M, et al. Clinical recovery from surgery correlates with single-cell immune signatures. Science translational medicine. 2014; 6: 255ra131
4. Rumer K K, Hedou J, Tsai A, Einhaus J, Verdonk F, Stanley N, et al. Integrated single-cell and plasma proteomic modeling to predict surgical site complications: A prospective cohort study. Annals of surgery. 2022; 275:582-59

Notwithstanding the appended claims, the disclosure set forth herein is also described by the following clauses:

1. A method for assessing a propensity to develop hemorrhagic transformation (HT) for an individual suffering from an ischemic stroke following an endovascular treatment (EVT), comprising:
obtaining a cellular biological sample for analysis comprising immune cells from a patient after having the acute ischemic stroke,
measuring single cell levels of activated signaling proteins in immune cell subset(s) in the immune cells;
determining whether levels of activated signaling proteins associated with propensity to develop hemorrhagic transformation are present; and
providing an assessment of the patient's prognosis for propensity to develop hemorrhagic transformation after the endovascular treatment.

2. The method of clause 1, wherein the cellular biological sample is a blood sample.

3. The method of clause 2, wherein the blood sample is an arterial or veinous blood sample.

4. The method of any of clauses 1-3, wherein the endovascular treatment is a thrombectomy or thrombolysis.

5. The method of any of clauses 1-4, wherein the obtaining occurs before the endovascular treatment.

6. The method of any of clauses 1-4, wherein the obtaining occurs after the endovascular treatment

7. The method of any of clauses 1-4, wherein the obtaining occurs before and after endovascular treatment.

8. The method of any of clauses 1-7, wherein the activated signaling proteins are associated with the mTOR pathway.

9 The method of any one of clauses 1-8, wherein the activated signaling proteins are selected from the group consisting of pSTAT1, pSTAT3, pSTAT5, pSTAT6, pERK, p38, pMK2, prpS6, pCREB, pNF-_{κ}B, total I_{κ}B and any combination thereof.

10. The method of any one of clauses 1-9, wherein the activated signaling proteins are prpS6 and pCREB.

11. The method of any one of clause 1-10, wherein the activated signaling proteins are prpS6, pCREB, pNF-κB, and pMAPKAPK2.

12. The method of any of clauses 1-11, wherein immune cells in the biological sample for analysis are gated for expression of cell surface markers.

13. The method of any of clauses 1-12, wherein the immune cell subset(s) are selected from the group consisting of CCR2-nonclassical monocytes, CCR2+ classical monocytes, CD56brightCD16-natural killer (NK) cells, CD56dimCD16+ NK cells, CD62L− aged neutrophils, CD62L+ immature neutrophils, CD66+ neutrophils, intermediate monocytes, myeloid dendritic cells, plasmacytoid dendritic cells, myeloid-derived suppressor cells, CD4+ central memory T cells, CD4+ effector memory T cells, CD4+ naive T cells, CD4+ resident memory T cells, CD8+ central memory T cells, CD8+ effector memory T cells, CD8+ naive T cells, CD8+ resident memory T cells, gamma-delta T cells, natural-killer T cells, memory T helper 1 (Th1) cells, naive Th1 cells, memory regulatory T cells, and naive regulatory T cells.

14. The method of any of clauses 1-13, wherein the immune cell subsets are memory Th1 CD4+ T cells and naïve Th1 CD4+ T cells.

15. The method of any of clauses 1-14, wherein the immune cell subsets are memory Th1 CD4+ T cells, naïve Th1 CD4+ T cells, CD66+ neutrophils, B cells, and non-classical monocytes.

16. The method of any of clauses 1-15, further comprising measuring the frequency of one or more specific immune cell subsets.

17. The method of clause 16, wherein the specific immune cell subset is non-classical monocytes.

18. The method of clause 16 or 17, wherein the specific immune cell subset is non-classical monocytes and classical monocytes.

19. The method of any one of clauses 1-18, wherein measuring single cell levels of activated signaling proteins in immune cell subset(s) is performed by contacting the sample with labeled affinity reagents specific for the activated signaling protein.

20. The method of clause 19, wherein measuring is performed by flow cytometry.

21. The method of clause 20, wherein the label is fluorescent.

22. The method of clause 19, wherein measuring is performed by mass cytometry.

23. The method of clause 20, wherein the label is an isotope label.

24. The method of any of clauses 1-23, wherein the determining comprises integrating the results of the measuring step into a multivariate model that generates a score that indicates an individual's propensity to develop HT following EVT.

25. The method of clause 21, wherein the multivariate model is generated using a sparse machine learning algorithm trained on a dataset containing the levels of activated signaling proteins and the frequency of specific immune cell subsets from individuals who have had HT following EVT and from individuals who have not had HT following EVT.

26. The method of any of clauses 1-25, wherein increased levels of prpS6 in memory Th1 CD4+ T cells, increased levels of pCREB in naïve Th1 CD4+ T cells, and an increased frequency of non-classical monocytes is indicative that an individual has a propensity to develop hemorrhagic transformation.

27. The method of any of clauses 1-25, wherein increased levels of prpS6 in memory Th1 CD4+ T cells, increased levels of prpS6 in CD66+ neutrophils, increased levels of prpS6 in B cells, increased levels of pCREB in naïve Th1 CD4+ T cells, increased levels of pNF-κB and pMAPKAPK2 in non-classical monocytes and an increased frequency of non-classical monocytes and classical monocytes is indicative that an individual has a propensity to develop hemorrhagic transformation.

28. The method of any one of clauses 1-27, wherein treatment of the individual is made in accordance with the prognosis.

29. The method of any one of clauses 1-28, wherein if the individual is determined to have a propensity to develop hemorrhagic transformation an effective amount of an mTOR inhibitor is administered before, during or after the endovascular treatment.

30. The method of clause 29, wherein the mTOR inhibitor is administered before the endovascular treatment.

31. The method of clause 29, wherein the mTOR inhibitor is administered during the endovascular treatment.

32. The method of clause 29, wherein the mTOR inhibitor is administered after the endovascular treatment.

33. The method of any of clauses 29-32, wherein the mTOR inhibitor is selected from the group consisting of sirolimus, everolimus, temsirolimus, deforolimus, umirolimus, zotarolimus, and a rapalog.

34. The method of any of the preceding clauses, wherein the endovascular treatment is a thrombectomy.

35. The method of any of the preceding clauses, wherein the endovascular treatment is thrombolysis.

36. The method of clause 35, wherein the thrombolysis comprises administering an effective amount of a tissue-type plasminogen activator or a urokinase-type plasminogen activator intravenously.

37. The method of clause 35, wherein the thrombolysis comprises administering an effective amount of a tissue-type plasminogen activator or a urokinase-type plasminogen activator intra-arterially.

37. The method of clause 35 or 36, wherein the tissue-type plasminogen activator is selected from the group consisting of Altepase, Reteplase, and Tenecteplase.

38. The method of clause 35 or 36, wherein the urokinase-type plasminogen activator is urokinase.

1. A method for assessing a propensity to develop hemorrhagic transformation (HT) for an individual suffering from an ischemic stroke following an endovascular treatment (EVT), comprising:
obtaining a cellular biological sample for analysis comprising immune cells from a patient after having the acute ischemic stroke,
measuring single cell levels of activated signaling proteins in immune cell subset(s) present in the immune cells;
determining whether levels of activated signaling proteins associated with the propensity to develop hemorrhagic transformation are present; and
providing an assessment of the patient's prognosis for propensity to develop hemorrhagic transformation after the endovascular treatment.

2. The method of claim 1, wherein the cellular biological sample is a blood sample.

3. The method of claim 2, wherein the blood sample is an arterial blood sample or a veinous blood sample.

4. The method of claim 1, wherein the endovascular treatment is a thrombectomy or thrombolysis.

5. The method of claim 1, wherein the obtaining occurs before or after the endovascular treatment.

6. The method of claim 1, wherein the obtaining occurs before and after endovascular treatment.

7. The method of claim 1, wherein the activated signaling proteins are associated with the mTOR pathway.

8. The method of claim 1, wherein the activated signaling proteins are selected from the group consisting of pSTAT1, pSTAT3, pSTAT5, pSTAT6, PERK, p38, pMK2, prpS6, pCREB, pNF-_{κ}B, total I_{κ}B and any combination thereof.

9. The method of claim 1, wherein the activated signaling proteins are prpS6 and pCREB.

10. The method of claim 1, wherein the immune cell subset(s) are selected from the group consisting of CCR2-nonclassical monocytes, CCR2+ classical monocytes, CD56brightCD16-natural killer (NK) cells, CD56dimCD16+ NK cells, CD62L− aged neutrophils, CD62L+ immature neutrophils, CD66+ neutrophils, intermediate monocytes, myeloid dendritic cells, plasmacytoid dendritic cells, myeloid-derived suppressor cells, CD4+ central memory T cells, CD4+ effector memory T cells, CD4+ naive T cells, CD4+ resident memory T cells, CD8+ central memory T cells, CD8+ effector memory T cells, CD8+ naive T cells, CD8+ resident memory T cells, gamma-delta T cells, natural-killer T cells, memory T helper 1 (Th1) cells, naive Th1 cells, memory regulatory T cells, and naive regulatory T cells.

11. The method of claim 1, wherein the immune cell subsets are memory Th1 CD4+ T cells and naïve Th1 CD4+ T cells.

12. The method of claim 1, further comprising measuring the frequency of one or more specific immune cell subsets.

13. The method of claim 12, wherein the specific immune cell subset is non-classical monocytes.

14. The method of claim 1, wherein measuring single-cell levels of activated signaling proteins in immune cell subset(s) is performed by contacting the sample with labeled affinity reagents specific for the activated signaling protein.

15. The method of claim 14, wherein measuring is performed by flow cytometry and the label is fluorescent.

16. The method of claim 14, wherein measuring is performed by mass cytometry and the label is a metal isotope.

17. The method of claim 1, wherein the determining comprises integrating the results of the measuring step into a multivariate model that generates a score that indicates an individual's propensity to develop HT following EVT.

18. The method of claim 17, wherein the multivariate model is generated using a sparse machine learning algorithm trained on a dataset containing the levels of activated signaling proteins and the frequency of specific immune cell subsets from individuals who have had HT following EVT and from individuals who have not had HT following EVT.

19. The method of claim 1, wherein increased levels of prpS6 in memory Th1 CD4+ T cells, increased levels of pCREB in naïve Th1 CD4+ T cells, and an increased frequency of non-classical monocytes is indicative that an individual has a propensity to develop hemorrhagic transformation.

20. The method of claim 1, wherein treatment of the individual is made in accordance with the prognosis.
